(19) United States
(12) Patent Application Publication
(10) Pub. No.: US 2006/0079752 A1
(43) Pub. Date: Apr. 13, 2006

(54) System for providing situation-dependent, real-time visual support to a surgeon, with associated documentation and archiving of visual representations

(75) Inventors: Horst Anderl, Effeltrich (DE); Artur Raczynski, Nurnberg (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengesellschaft

(21) Appl. No.: 11/234,348

(22) Filed: Sep. 23, 2005

(30) Foreign Application Priority Data
Sep. 24, 2004 (DE) 10 2004 046 430.8

(51) Int. Cl. A61B 5/05

(52) U.S. Cl. 600/407

(57) ABSTRACT

A system for providing the situation-dependent real-time visual support to a surgeon during a surgical operation, and for real-time documentation and archiving of the visual impressions generated by the support system and perceived by the surgeon during the operation, has a visualization device for outputting data, to the surgeon during the surgical operation, a first video camera fixed to the visualization device, at least one second video camera focusing on the operating area from an angle of view that is different from that of the video camera of the visualization device, at least one central computing unit that is connected to the visualization device, to the video cameras, to operation monitoring components, and to computing devices, via a medical information system and the central computing unit has a front end merger that controls the visual presentation of data of the visualization device.

### BACKGROUND OF THE INVENTION

1. Field of the Invention

The present invention concerns a system for providing visual support to a surgeon during an operation, in particular with the use of visualization devices worn at the head, or head-mounted displays (HMD). The present invention in particular concerns a system for real-time visual support, as well as real-time documentation and archiving of the surgeon's visual impressions.

2. Description of the Prior Art

There are various ways in which a surgeon can be provided with visual support in the operating room (called OP in the following); their expense varies according to the manner of their construction. In a comparatively modern system (FIG. 1, explained in more detail below), the OP is equipped with a number of modalities 1 (e.g., x-ray C-arm, ultrasound, MRT), as well as with (a number of) video cameras 2 and one or more monitors, and sometimes with patient monitoring devices (e.g. ECG, EEG, etc.), each of which are connected to an OP computer 4. In addition, the surgeon 13 wears, in front of his or her eyes, an eyeglass-type visualization device 5 that is provided with a video eyeglass camera 3 that can be oriented in the direction in which the surgeon is looking. The two optical systems—visualization device 5 and video eyeglass camera 3—are also connected to OP computer 4, either by a cable or wirelessly.

Various technologies are used as the visualization device, e.g. a laser fixed in front of the eyes of the surgeon 13 that projects virtual data in the form of a virtual display onto the retina of the surgeon, there producing a virtual user interface (UI). This technology is known for example under the name “retina scanning display” (RSD), made by the firm Microvision. Other technologies include, for example, mini-displays that can be worn at the head of the surgeon and that are integrated either into eyeglasses (operations microscope OPMI Pencho, by Zeiss) or that provide virtual images in front of the surgeon's eyes (head-up display). In all cases, the produced display, which represents data in the visual field of the surgeon, follows the surgeon's head movements; for this reason, all described specific embodiments of the visualization device in the following are designated as head-mounted displays, or HMD. Additional visualization devices can include an operation microscope, or simply a central display device that is suspended over the OP table.

The visual support for the surgeon 13 in the OP takes place by the support system generating (image) data and displays the data in the HMD 5 of surgeon 13. The preparation for this is carried out before the operation at OP computer 4 by medical personnel or by surgeon 13.

For simpler visual support systems, for example if differentiated data are presented exclusively on different monitors, a coordination of the surgical interventions based on image navigation in the prepared images (e.g. using the mouse, using voice-recognition software, using foot movements, etc.) and image analysis can take place only in sequential fashion, because during the operation the surgeon has to change his or her direction of view, so that the area of the operation is no longer in his or her visual field.

Visual support using HMD 5 makes it possible for surgeon 13 to represent data (image data, information data, signals, etc.) in such a way that his or her required attention during the operation is not diverted.

Currently, the surgeon 13 sees, through the use of an HMD 5 via an OP computer 4 or via one or more computers situated inside or outside the operating room, available patient images that have been acquired a relatively short time before the operation—sometimes via various modalities 1—and that may have been post-processed on the respective computer (modalities computer, image workstation computer) in such a way that they enable the surgeon 13 to find an optimal anatomical orientation during the operation. It is already technically possible to present a number of image data sets alongside one another in different windows, or in overlapping fashion, using the HMD 5. Analysis functions and navigation means are also available to the surgeon 13 in order to optimally adapt the representation of data on the HMD 5 to the surgeon's needs.

A visual OP support system with HMD according to the prior art is shown in FIG. 1A in roughly schematic fashion. A central OP computer 4 has at least one front end 6 (also called a graphical user interface or GUI) via which, during preparations for the operation, HIS data (“Hospital Information System”), RIS data (Radiological Information System), and PACS data (Picture Archiving and Communication System) are coordinated and are correspondingly displayed using a visualization the device (HMD) 5. During the operation, it is also possible for images (tomograms, 3-D data sets) recorded using different modalities 1 (CT, MRT, US) to be received, in unmodified form or in post-processed form, in the (or in a) front end 6, and played back in the HMD 5. The front end 6 can be regarded as a computing unit or as a configuring software component that controls the data representation by the HMD 5. The OP computer 4 additionally has what is known as a back end 7, via which the OP computer is connected to the HIS, RIS, and PACS data source (e.g., hard drive data file, database, etc.), and via which all image data involved in the operation can be archived for the purposes of teaching, demonstration, documentation, or reproduction. Involved image data include images or films from all video cameras 2 situated in the OP (e.g., camera 2 in FIG. 1), including video eyeglass camera 3 (HMD camera, camera 1 in FIG. 1), all unmodified and post-processed modality images recorded during the operation, and the overall sequence presented visually to the surgeon 13 during the overall operation by the HMD 5. The back end 7 is also a computing unit, and can be regarded as an administrative unit for the connected data sources.

FIG. 1B shows, in a highly simplified fashion, the functioning of an HMD-supported visualization system in the OP surgery ward, according to the prior art. Relevant data (for example KIS, RIS, PACS data) are loaded via back end 7 into the respective front end (Graphic User Interface, GUI) 6, where they can then be visualized in a specific software environment. The choice of which data are finally displayed, for example on an HMD, by means of the visualization device, i.e., which front end 6 is finally put into active use, is made by the surgeon 13 by actuation (e.g. mouse-based) of a switch.

The modern visual support system as described above and as shown schematically on the basis of FIGS. 1A and 1B has various disadvantages:

1. All the data that are supposed to be displayed via the HMD (generally referred to as the visualization device in the following) must be previously calculated, i.e. prepared, on the OP computer or on a computer connected to the OP computer before or during the operation. The preparation relates to the selection of the data and thus to the number of data fields that are to be displayed, the display size and resolution of the respective data field, etc. At least in the context of a relatively large time window, the surgeon is limited to the information present on the OP computer. If a situation arises that was not foreseeable, it is not possible for the surgeon, using a currently available visual support system, to immediately be provided with a visual display on the visualization unit of additional information not provided by the back end or front end. Indications coming from the outside, for example from expert colleagues who are not “on location” and who are following the progress of the operation, can be communicated only acoustically or on a separate monitor, causing the surgeon to interrupt his working process.

2. It is currently not possible to carry out a real-time reproduction-time synchronization, or a documentation synchronization. That is, it is currently not ensured that the data visualized using the visualization device precisely describe the identical physical situation at the current point in time, which has the further result that the data archived by the back end are not documented in chronologically synchronous fashion, and thus that the actual sequence of events in the operation cannot be precisely reproduced.

The latter is important in the case of an operation having an undesired outcome, in which case a reproduction of the course of the operation including all data (information) that was given visually to the surgeon can be presented in court, and can serve to determine if an error occurred.

3. In a current visual support system, the surgeon can lose visual contact with the real image of the region being operated on, for example if the open wound of the body is obstructed by his or her own hands or by instruments, forcing the surgeon to divert his or her gaze and to pursue the surgical intervention only indirectly on the monitor, via an additional camera situated in the OP that in this case offers the best view of the open part of the body.

4. The preliminary calculation and archiving of data in current visual support systems represents a redundant storage of data from a clinical point of view, because currently each image is stored completely (e.g. in DICOM format) on the OP computer, or the complete film from each individual video camera is stored on the OP computer.

5. The data to be visualized on the HMD or on some other visualization device can currently be played only in their respective visualization software environment (i.e., in a window of the respective front end) in the visualization device, which has the result that apart from the image or signal curve that is of interest, the corresponding graphic user interface overlay of the respective front end must also be displayed, thus occupying valuable memory space of the visualization device with a large number of elements (buttons, menu bars, scroll bars, etc.).

### SUMMARY OF THE INVENTION

An object of the present invention is to achieve a system and a computer software product for providing visual support to a surgeon in the OP that avoid the aforementioned problems and that provide a solution for visualization device-based support according to the needs of the situation, and that make this support capable of being influenced in real time.

According to the present invention this object is achieved by a system that provides situation-dependent real-time visual support to a surgeon during an operation, as well as for real-time documentation and archiving of the visual impressions generated by the support system and perceived by the surgeon during the operation, having a visualization device (e.g. an HMD) for outputting data, to the surgeon during the surgical operation a first video camera fixed to the visualization device, oriented in the direction of view or direction of the head of the surgeon, at least one second video camera focusing on the operating area from an angle of view that is different from that of the video camera of the visualization device (showing the point of the operation from a different direction of view), at least one central computing unit connected to the visualization device, to the video cameras, to operation monitoring components, and to computing devices via a medical information system, and wherein the central computing unit has a front end merger that controls the visual presentation of data of the visualization device.

According to the present invention, if obstructing objects are present in the field of view of the first camera, the front end merger switches to the second camera in order to acquire an unobstructed view of the operating site, and represents this in the form of an output of the visualization device.

In addition, the central computing unit, or one of the computing devices, has a back end merger that, relative to a central fixed system time clock, centrally and/or decentrally archives the chronological sequence of the visual representation of the data by the visualization device.

According to the present invention, a decentralized archiving takes place based on administration of time-marked reference addresses.

The system according to the present invention preferably is designed such that the back end merger creates a documentation document that contains the time-marked reference addresses of the output data and/or contains the time-marked data themselves that are output via the visualization device.

In addition, according to the present invention the front end merger effects a chronologically synchronized representation of the data output by the visualization device.

According to the present invention, the decision as to which data are to be output by the visualization device at what point in time, relative to the system time, is made by the front end merger on the basis of a decision table.

The front end merger also can influence the manner of presentation of the data outputted by the visualization device.

According to the present invention, the data output by the visualization device originate from
current or archived original or post-processed data of various imaging modalities, and/or
physicians or experts observing the operation from an external location.

According to the present invention, the visualization device is formed
as a display device mounted at the head of the user, and/or
as a fixed laser that projects data into the retina of the user, and/or
as an operation microscope and/or
as a central display device suspended over the OP table.

The front end merger effects the display of the data that are of interest exclusively in the form of images, signals, signal curves, etc., and suppresses the display of the graphic user interfaces (overlays) of the respective image-generating or signal-generating graphic user interface.

The aforementioned additional operation monitoring components include, for example, one or more of an ECG apparatus, an EEG apparatus, a blood pressure measuring device, a laparoscope, an endoscope, a device for monitoring respiration, an operation microscope, etc.

In addition, an advantageous and basic feature of the present invention is that the data output by the visualization device are called via an information network as HIS data, RIS data, SAP data, or PACS data, and are made available in real-time.

The above object also is achieved according to the present invention a computer software product (a storage medium encoded with computer-readable information) that programs a computerized system to enable functioning of the system in the manner described above.

### DESCRIPTION OF THE DRAWINGS

FIG. 1A schematically shows the interaction of components of an HMD-supported visualization system in the OP surgical ward according to the prior art.

FIG. 1B schematically shows the data source connection of an HMD-supported visualization system in the OP surgical ward according to the prior art.

FIG. 2 schematically shows an HMD-supported visualization system according to the present invention in an OP surgical ward.

FIG. 3A schematically shows the data source connection of an HMD-supported visualization system according to the present invention in the OP surgical ward.

FIG. 3B schematically shows the functioning, or the interconnection of the components, of the visualization system according to the present invention.

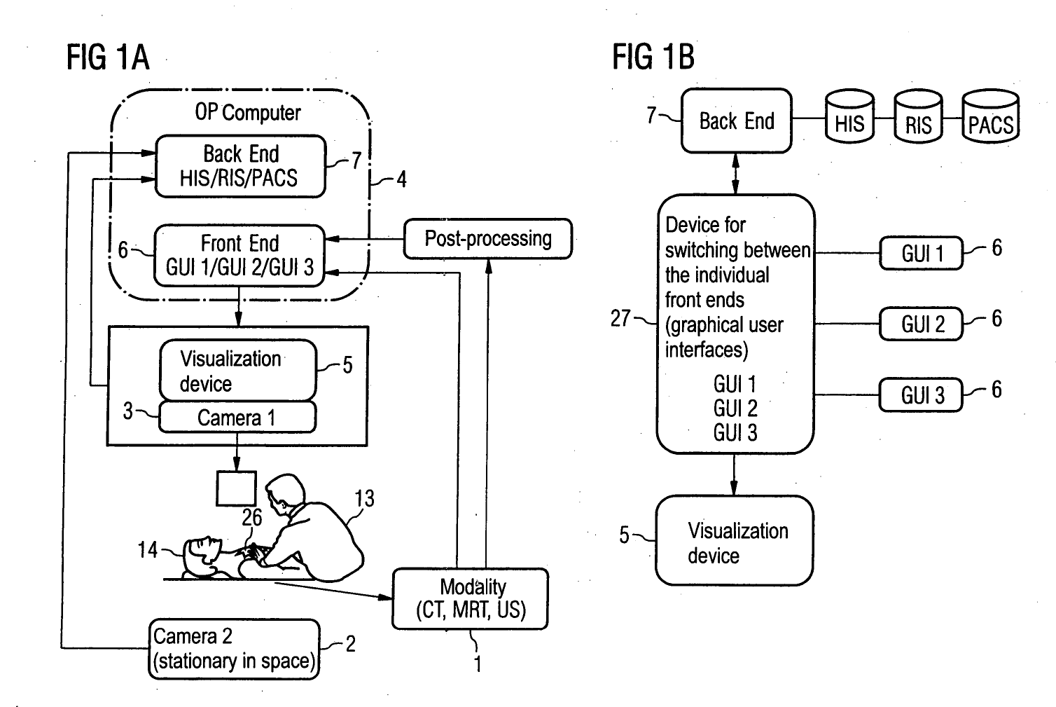

### DESCRIPTION OF THE PREFERRED EMBODIMENTS

The present invention is designed to provide situation-adapted (real-time) presentation of data and/or of information to the surgeon in the context of a visual support system in the context as described in detail above, as well as to provide chronologically synchronized archiving of all called or visualized data. For this purpose, according to the present invention OP computer 4 is expanded by the creation of two new interfaces 10, 11, via which the OP computer communicates online with internal and/or external data sources and with other external computers that supply data, so that OP computer 4 becomes a real-time-capable OP central computer 9. Additional external computers that supply data include for example PCs or (micro)processors of HMDs, (video) cameras, modalities, and image processing stations that are connected to the OP computer (or to the computer or computers inside or outside the operating room, henceforth called the OP central computer 9) via hospital-internal networks, or via worldwide (medical) networks (HIS, RIS, PACS, the Internet, etc.).

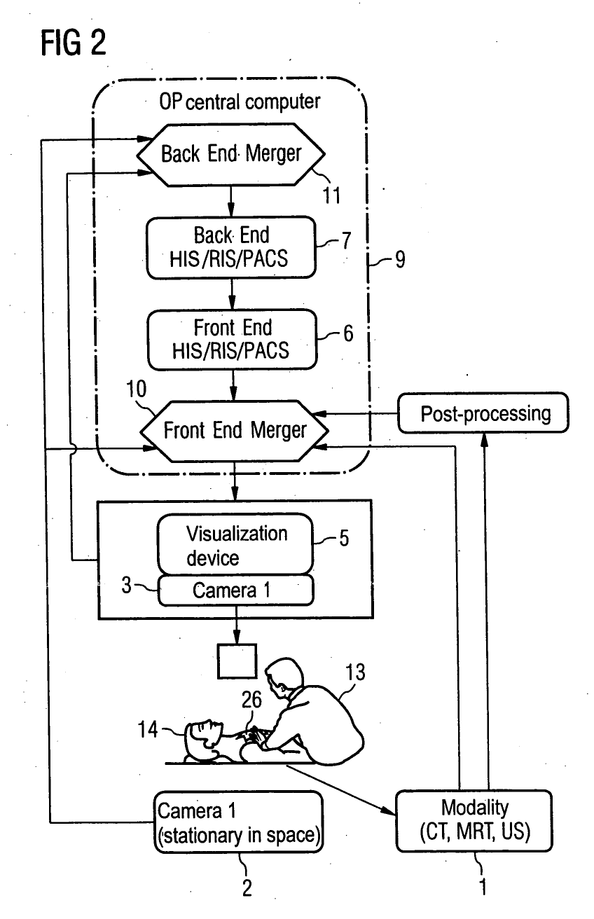

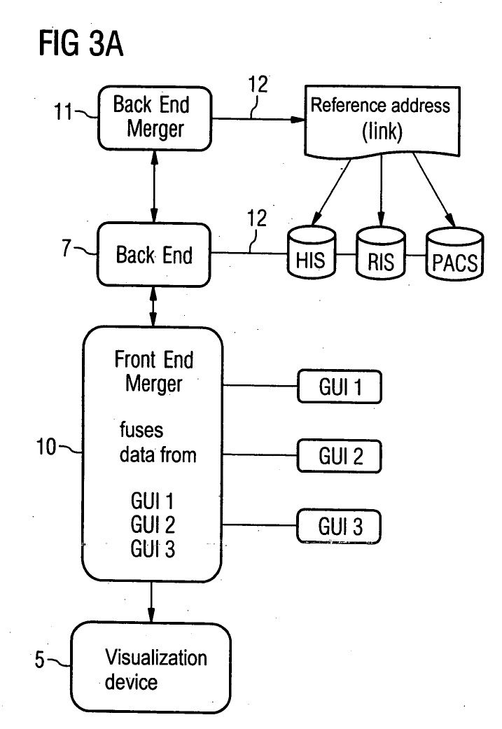

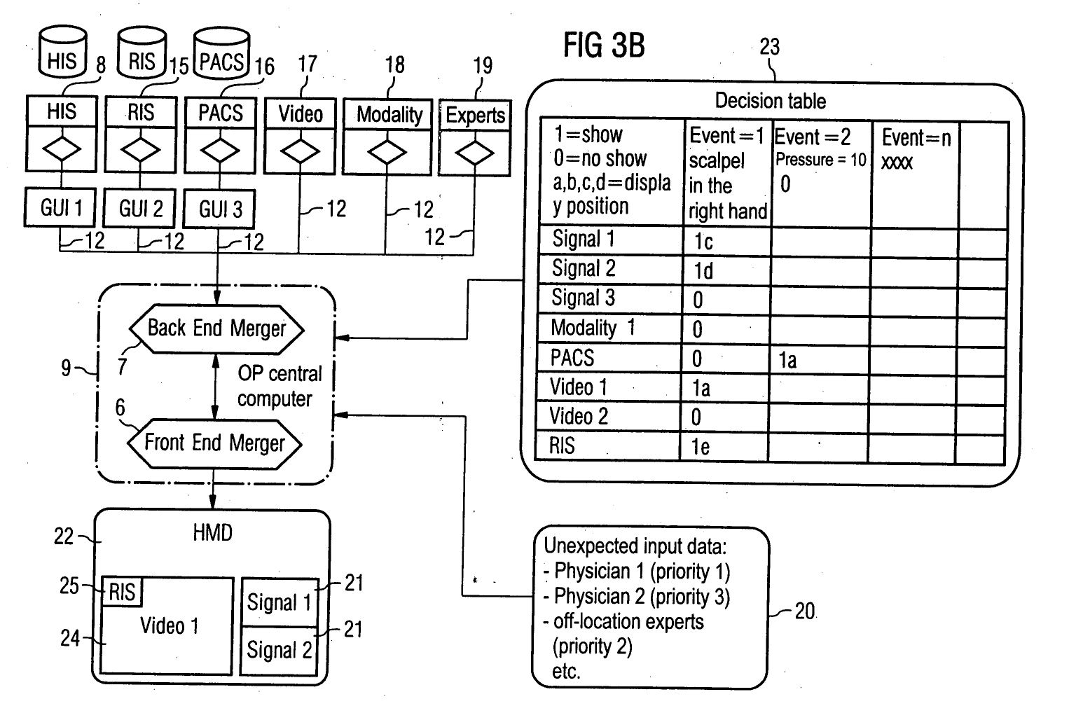

The first of the two interfaces is used for the online provision of information by means of a visualization device (HMD) 5, and is designated front end merger 10, because this module merges exclusively the data (image, signals, etc.) that are currently of interest from various front ends (e.g., HIS data GUI 1, RIS data GUI 2, PACS data GUI 3, etc.) on the HMD 5 in fused fashion or adjacent to one another or in overlapping fashion, as is shown in FIGS. 2, 3A, and 3B.

The second of the two interfaces is used for an optimized chronologically synchronized documentation or archiving of all data visualized by means of visualization device (HMD) 5, and is designated back end merger 11, because this module is connected preceding the back end 7, as is also shown in FIGS. 2, 3A, and 3B.

The two features cited above (front end merger and back end merger) are connected to one another inside the computer, either via the back end 7 or via front end 6 are connected directly, so that ultimately the manner in which the input data from external computers are distributed to the two interfaces and processed inside the computer depends on the software architecture of the OP central computer 9.

Through the configuration according to the present invention of the OP central computer 9 by means of the back end merger 11 and the front end merger 10 and their points of network access 12, as shown in FIG. 3B, access data can be requested during the operation either by the surgeon 13 or by another person participating in the operation (assistant, anesthesiologist, MTA, etc.), via the OP central computer 9, and displayed in the visualization device (HMD) 5. Such input data, for example (see FIG. 3B) are archived HIS data 8, RIS data 15, PACS data 16, video images or films 17 from various cameras (for example from previous laparoscopic examinations), or current images from various modalities 18 (ultrasound, x-ray C-arm, etc.).

Additional input data can be text data and sound data or marked image data through which off-location experts 19, or for example an external team of physicians 20 (physician 1, physician 2), following the operation via video, can spontaneously express themselves in order to provide assistance to operating surgeon 13 via the visualization device (HMD) 5.

In addition, input data can be current important physical values or signal curves 21 (FIG. 3B: signal 1, signal 2) that represent devices monitoring the patient's state (blood pressure, ECG, EEG, respiration, etc.).

The possibilities for access, which are increased according to the present invention, to this wealth of possible input data, available in an easily surveyable, chronologically synchronized representation by the visualization device (HMD) 5, as well as a chronologically synchronized archiving of the data actually presented visually to surgeon 13 during the operation, require an extensive expansion of the visualization system or the implementation of a series of (software) components in the OP central computer 9, described in detail in the following:

A) From the pool of available information (data, input data), data must be prioritized according to medical importance in a manner adapted to the particular situation, and must be selectively represented by the visualization device (HMD) 5, not only to avoid an overload of information, and thus a lack of surveyability of the image of the visualization device (HMD image) 22, but also to achieve an optimal, useful, chronologically synchronized representation, corresponding to the current situation, with respect to position, size, resolution, etc.

The module that enables this is the front end merger 10. The front end merger 10 makes a selection from the available, requested, or present data on the basis of a decision matrix 23 (see decision table 23 in FIG. 3B), which is defined in accordance with predetermined rules for prioritizing the access sequence (in the context of a given network bandwidth and server performance level).

The decision matrix 23, which influences and regulates the data access and thus the representation by visualization device (HMD representation) 22, is configured by the manufacturer during the installation of the OP central computer 9 in consultation with the user (OP team), and is therefore dependent not only on the case of application (type of operation, type of intervention), but can vary greatly from hospital to hospital, or even from OP team to OP team. This is because, depending on the available technology and the degree of experience of the OP team, the same surgical intervention can follow a very different course from one case to the next. It may also occur that operating surgeon 13 may be confronted with a new, unforeseeable situation (for example, necessary or unintended damage to an organ or to a blood vessel). In such a case, it is necessary to keep the rule description capable of being modified in real time in order to give the surgeon 13 the possibility, for example, of changing the execution rights, requesting external assistance, adding patient monitoring data 21 to the display, switching to a different video camera 2, etc.

The ability to flexibly control of the visualization device (HMD) 5 means that it is necessary not only to carry out a reconfiguration of the image of the visualization device (the HMD image) 22 at front end merger 10; but also this new image arrangement must be adapted to new resolution requirements by communicating the target resolution to a resolution converter. Such a resolution adaptation algorithm can be coupled with a segmentation method which, according to the situation-dependent data request, represents only the partial segment that is important at that moment for the surgeon 13 (e.g., only blood vessels, or only bone tissue of a CT exposure).

B) The fused data added in on visualization device (HMD) 5 during a visually supported operation should be archived in chronologically synchronized fashion for documentation purposes.

The module that enables this is the back end merger 11.

In the context of an archiving process, the back end merger 11 applies a data container data file (a documentation document) that documents which data set was visualized by the HMD 5 at what time, and for how long, during the operation.

For this purpose, each displayed data set is registered under an information identification number (ID) that is provided with additional attributes and is stored in the data container data file. Such attributes include a time stamp that is coupled to an OP-central clock time, and that for example is assigned by an agent of the data source during the data transmission (routing), as well as an attribute relating to whether and where an item of information was displayed (by the HMD 5 or on another monitor in the OP), and an attribute relating to whether, when, and by whom this information (this data set) was suppressed.

In the case of an operation using modalities, as an attribute entry the DICOM specifications can be used for all data sets coming directly from modality 1, 18, if the date of generation and the time of provision (viewing date) are the same. DICOM (Digital Imaging and Communication) standardizes the structure of the formats and descriptive parameters for radiological images, as well as commands for exchanging these images, and has a field in which the date of generation is entered. For data sets that come from PACS, it is currently not possible to register the viewing date. For data sets called from information systems such as HIS, RIS, or SAP, it is possible to assign the time stamp via the audit trail of an additional standard, namely HL7. A goal of the patent is to store this time stamp as well (the format in which it is stored is not important here).

The information as to which monitor or HMD the respective data set appeared on, and whether this data set was actively or automatically suppressed, can currently be stored neither in DICOM nor in HL7. If the attributes (time stamp, display location, etc.) cannot be managed in the data set data file (the information file) itself, it is however possible to store these in the already-mentioned data container data file. It should be noted that an attribute assignment can be circumvented only via a data container data file, by a possible expansion of the system standards (DICOM, HL7, etc.). It also makes sense to avoid memory redundancy by having the data container data file contain only references (or reference addresses, i.e. links; these indicate which data set was visualized when and where); i.e., the data set itself is not stored a second time, but rather occurs only once in all the information systems.

Also for reasons of archiving space, but also for reasons of surveyability, according to the present invention the front end merger 10 and the back end merger 11, or a combination of the two, realize an automatic mechanism for producing from the video streams of a plurality of video cameras a single video that is ultimately archived. According to the present invention, the logic of the selection between the various video streams is realized in such a way that the archived video predominantly shows the unobstructed view of the surgeon 13.

If the open site of the body is obstructed by the hands of the surgeon 13 or by an instrument (obstruction situation), then, taking into account defined rules, a view that is best for the observer is generated and is displayed at the surgeon's visualization device (HMD) 5. In the context of the present invention, these rules are defined in operation-specific manner, or in a manner related to the team carrying out the operation.

On the basis of two sample cases that could actually occur in the OP, it is now explained how the system according to the present invention reacts in interaction with the persons taking part in the operation (see FIG. 3B):

Case 1: In an OP situation, the surgeon's view of the open part of the body is obstructed by the surgeon's hands. In the visualization device (HMD) 5, there automatically appears an image 24 from a camera (video 1) that for this case offers the best view of the open site of the body. While the surgeon 13 orients himself or herself in the open body site with the aid of video image 24 (video 1), in the visualization device (HMD) 5 the surgeon 13 is additionally supplied, via the radiological information system, with the tomograms originating from a CT exposure (for example in the upper left corner 25 RIS of the display). The tomograms are oriented, for example, to the tip of his scalpel. In this way, he can pursue his intervention in 3-D without losing sight of the real image. In addition, on the right side of the HMD there appear, in a vertical orientation, measurement values of patient 21 (signal 1: ECG; signal 2: blood pressure), confirming his correct intervention.

Case 2: After the operation, the supply of information during the operation is critically discussed in a meeting of the OP team. For this purpose, all data (information) presented on HMD 5, monitors, and device displays are discussed that were present when the pulse rate of patient 14 increased drastically at minute 10:01 after the beginning of the operation. An intervention by the surgeon 13 was successful in restoring the normal state of patient 14 at minute 23:05. For this purpose, the documentation system supplies all data (information) from minute 10:01 to minute 23:05. The data are displayed on monitors, in the same way in which they were perceived by surgeon 13 during the operation via his visualization device (HMD) 5. In addition, the system displays—possibly distributed among several monitors—all data that were available at this time or during this critical time window. In response to this, the OP team changes the rules (decision matrix 23) of the HMD automatic mechanism in such a way that in future operations of this type the ECG values will additionally appear in a sub-area of the HMD when the physician is holding the scalpel in his or her right hand.

In FIG. 3B, it is shown that OP central computer 9 is supplied only with data that are also able to be visualized by the visualization device 5 (HMD) (HIS data, RIS data, PACS data, video, modalities, input from experts). What is known as an expert system (neural network, decision system, rule logic) in the OP central computer 9 is based on a decision table 23 that, dependent on the OP team (user profile), as well as on the type of operation, contains decision rules according to which the data visualization by visualization device (HMD) 5 takes place. The decision table 23 can be modified interactively during the operation. For this purpose, there is also a priority table 20 that assigns an order of rank to unexpected input data (for example, spontaneous pieces of advice coming from off-location physicians connected via video): physician 1 has first priority, physician 2 has third priority, etc. In addition, FIG. 3B shows that the components back end merger 11 and front end merger 10, provided according to the present invention, are what make possible a real-time combination between the HMD 5 and the data 15 to 19 that are available via the network 12. Through them, the OP central computer 9 not only controls the visualization (arrangement of data by HMD 5 with a suitable resolution), evaluates unexpected input data, and influences decision matrix 23, but also displays only synchronized data, by comparing the time stamps of all data sets. If, for example, an ultrasound video enters in a manner that is not synchronous with the EKG curve, a synchronization takes place through evaluation of the generation times. With the use of the already-existing synchronization standard CCOW, the purpose of which is to achieve synchronization between different applications at a single (image) workplace, the aspects of the above-mentioned documentation synchronization and reproduction time synchronization must, however, also be concomitantly recorded.

The knowledge of the generation times is based on a suitable agent software that is implemented in the computers (source computers) 8, 15 to 19 that supply OP central computer 9, or in device processors, and that tells the OP central computer 9 whether and when an item of information is to be handed over, whereby the required computing power and network transmission rate are likewise kept available. The agent software of each source computer 8, 15 to 19 knows the boundary values of the decision table 23, and sends current data to the OP central computer 9 only if these data are relevant in the context of the decision rules, or if they become relevant in this context. For this purpose, the agents of the source computers create a network connection 12 (routing) and transmit only the data requested on the basis of the decision table. The communication as to which signals are concerned is supplied by the OP central computer 9 at the decisive moment to the agents of the respective source computer 8, 15 to 19.

FIG. 3A shows the functioning according to the present invention, or a possible combination of components according to the present invention, of the visualization system according to the present invention again in an overview:

The back end merger 11 and the front end merger 10 are connected to one another, for example, via the back end 7. The back end 7, and also the back end merger 11, have access, e.g. via networks 12, to HIS, RIS, and PACS data that, on request, are prepared by specific front ends and are correspondingly provided in real-time in the context of various front ends (GUI 1, GUI 2, GUI 3). Front end merger 10 exports the images and signal curves of the GUIs on the basis of a decision table, or on the basis of a control mechanism, and fuses them on the visualization device (HMD) 5 in side-by-side and/or overlapping fashion. The back end merger 11 creates a documentation document in which all reference addresses (links) are archived with the time stamp of the actually visualized data of the visualization device 5 (HMD data), so that the visualization that actually took place can be precisely recreated at an arbitrary later time on the basis of the documentation document.

In sum, the visual support system according to the present invention has the following advantages:
The quality of the operation is improved by the possibility of calling arbitrary (not specifically prepared) data, and of optimally displaying these data, during the operation (information on demand).
A chronologically synchronized documentation of the overall operation is enabled.
The content of the documented data is improved or optimized in that the video segments of various cameras are selected.
The video data of the cameras can be archived together with HIS, RIS, PACS, SAP data in a common archive.
The archiving of reference addresses (links) in container data files means that fewer data have to be archived.
Because data (information) is displayed only in a situation-dependent manner, the network load in the medical (OP) network is comparatively low.

Although modifications and changes may be suggested by those skilled in the art, it is the intention of the inventors to embody within the patent warranted hereon all changes and modifications as reasonably and properly come within the scope of their contribution to the art.

We claim as our invention:

1. A system for providing situation-dependent, real-time visual support to a surgeon during a surgical operation conducted at a surgical site, comprising:
a visualization device that emits information, perceivable by the surgeon, concerning the surgical operation;
a first video camera affixed to said visualization device and having a first field of view of the surgical site, said first video camera generating first image data representing said surgical site from said first field of view;
a second video camera having a second field of view of the surgical site, different from said first field of view, said second video camera generating second image information representing said surgical site from said second field of view; and
a central computing unit having a front end merger having an input connected to said first video camera for receiving said first image data therefrom, and an input having access to said second image data as well as additional data from at least one additional source, said front end merger controlling which of said first image data, said second data and said additional data is included in said information emitted to the surgeon by said visualization device.

2. A system as claimed in claim 1 wherein said front end merger normally causes said first image data to be included in the information supplied to the surgeon by said visualization device and, if an obstruction to said surgical site occurs in said first field of view, said front end merger automatically substituting said second image data, in place of said first image data, in said information emitted to the surgeon by said visualization device.

3. A system as claimed in claim 1 wherein said central computing unit comprises a back end merger having an output at which a chronological sequence of said information emitted to the surgeon by said visualization device is emitted for archiving, and an electronic archive connected to said output of said back end merger in which said chronological sequence is stored.

4. A system as claimed in claim 3 wherein said memory comprises a decentralized archiving system having a plurality of memory locations, and wherein said back end merger correlates said chronological sequence with time-marked reference addresses, with portions of said chronological sequence being respectively stored at said different memory locations dependent on said addresses.

5. A system as claimed in claim 4 wherein said back end merger generates a documentation document containing at least one of said time-marked reference addresses and information in said chronological sequence marked by said time-marked reference addresses.

6. A system as claimed in claim 1 wherein said front end merger chronologically synchronizes said information emitted to the surgeon by said visualization device.

7. A system as claimed in claim 6 wherein said front end merger comprises an electronic decision table providing criteria for organizing said information in said chronologically synchronized presentation relative to a system clock.

8. A system as claimed in claim 1 wherein said front end merger controls a presentation format of said information emitted to the surgeon by the visualization device.

9. A system as claimed in claim 8 wherein at least some of said first image data, said second image data and said additional data comprise a graphic user interface overlay, and wherein said front end merger suppresses presentation of said graphic user interface overlay in the information emitted to the surgeon by said visualization device.

10. A system as claimed in claim 1 wherein said second input of said front end merger is adapted to receive said additional data as data selected from the group consisting of current data from at least one imaging modality, archived data from at least one imaging modality, and post-processed data from at least one imaging modality.

11. A system as claimed in claim 1 wherein said second input of said front end merger is adapted to receive said additional data from at least one component that monitors said surgical operation, as said additional data source.

12. A system as claimed in claim 1 wherein said second output of said front end merger is adapted to receive said additional data from at least one person observing said surgical operation from a location remote from said surgical site, as said additional data source.

13. A system as claimed in claim 1 wherein said visualization device is selected from the group consisting of display devices adapted to be worn on the head of the surgeon, and laser devices that project image data onto a retina of the surgeon.

14. A system as claimed in claim 1 wherein said visualization device comprises a surgical microscope.

15. A system as claimed in claim 1 wherein said visualization device comprises a central display device mounted over said surgery site.

16. A system as claimed in claim 1 wherein said second input of said front end merger is adapted to receive said additional information from said additional data source selected from the group consisting of an ECG apparatus, an EEG apparatus, a blood pressure monitor, a laparoscope, an endoscope, a respiration monitor, and a surgical microscope.

17. A system as claimed in claim 1 wherein said central computer comprises a back end merger, having an output connected to said second input of said front end merger, said back end merger having an input selectively connectible to at least one of a source of HIS data, a source of RIS data, a source of SAP data and a source PACS data, as said additional data source, and wherein said back end merger makes said additional data available in real-time.

18. A storage medium encoded with a computer-readable program, said storage medium being loadable into a computerized apparatus having a front end merger having an output connected to a visualization device that emits information concerning a surgical site to a surgeon conducting a surgical procedure at the surgical site, and having a first input that receives first image data from a first video camera affixed to the visualization device, said first video camera having a first field of view of said surgical site, and having a second input adapted to receive second image data from a second video camera having a field of view, different from said first field of view, of said surgical site, and additional information associated with said surgical site from at least one additional information source, said program operating said front end merger to control which of said first image data, said second image data and said additional information are included in the information supplied by the front end merger to the visualization device to be emitted by the visualization device the surgeon.
